(19) United States
(12) Patent Application Publication
(10) Pub. No.: US 2014/0363504 A1
(43) Pub. Date: Dec. 11, 2014

(54) ABSORBABLE ENCAPSULATED CALCIUM IN HYPROMELLOSE CAPSULE

(71) Applicant: The Beauty Nation Ptd Ltd, Jurong (SG)

(72) Inventor: Tan See LENG, Jurong (SG)

(73) Assignee: The Beauty Nation Ptd Ltd, Jurong (SG)

(21) Appl. No.: 14/256,405

(22) Filed: Apr. 18, 2014

(30) Foreign Application Priority Data
Jun. 11, 2013 (SG) 201304495-3

(51) Int. Cl. A61K 9/48; A61K 31/4439; A61K 31/616; A61K 33/06

(52) U.S. Cl. CPC A61K 9/4816; A61K 33/06; A61K 31/4439; A61K 31/616; A61K 9/4891
USPC 424/457; 424/682

(57) ABSTRACT

An absorbable encapsulated calcium in hypromellose capsule, characterized in that the encapsulated calcium comprises calcium amino acid chelate extracted from soya bean, aspartate, calcium amino acid chelate which is obtained from chelation of aspartate with calcium hydroxide, collagen, lecithin, soya bean extract, lactose, calcium carbonate, in the ratio of 4:8:8:10:8:12:10:40 being capsulated into hypromellose capsule capable of being absorbed in vivo at the small intestine beyond the stomach, which maximize human absorption of calcium.

### FIELD OF THE INVENTION

The present invention relates to the field of oral pharmaceutical nutrient or a targeted release nutrient. More particularly, the present invention relates to an encapsulated calcium salt which provides effective in vivo absorption in the small intestine of humans.

### BACKGROUND OF THE INVENTION

Focus is drawn to the question of supplementing our diets with calcium and why the consumed calcium is wasted. In other words why so much of the calcium that consumed is wasted. Normally, we only absorb about 10% of the calcium we ingest. Accordingly, the impact of focus on calcium absorption may therefore be more profitable for those who are concerned about maintaining and building healthy bones. So, the accurate time of release properties that can help the consumed calcium to be released at a specific place of the body is important and is required to be studied.

The factors that influence calcium absorption in the human body are three major regulators of calcium metabolism in the body. The parathyroid glands produce a hormone (PTH) which moves calcium from the bones into the bloodstream. It also signals the kidneys conserve calcium and other minerals from the urine. Additionally, PTH signals the kidneys to produce calcitriol, which is formed from vitamin D, and which, among other functions, signals the small intestine to absorb more calcium. The thyroid gland secretes calcitonin, which increases bone mineralization, and decreases the rate at which the bone is broken down.

What is wanted to ameliorate the lacking of calcium is to increase calcium intake by supplying sufficient amount of calcium to the body. There are plenty of calcium-containing pharmaceuticals and calcium-enriched foods are available in the markets of today, they are inferior in palatability and difficult to take habitually.

Under these circumstances, it is important to exploit orally taken calcium be absorbed efficiently through the intestine, which is the absorption site for calcium. It is considered that the ratio of calcium absorbed through the intestine and utilized for maintenance of a living body in the total calcium orally taken is about 20 to 50%, while somewhat varying depending on the origin of calcium, so that the most of orally taken calcium is excreted without being absorbed.

Therefore, an increase in percentage absorption of calcium in the intestinal tract would fulfil the calcium requirement without forcing unnatural calcium intake.

It has been a known knowledge that absorption of oral intake of calcium is at maximum at small intestine, and accordingly, it is the object of the present invention to provide an encapsulated calcium for oral intake for which the pharmaceutical calcium be absorbed when the capsule arrives at the small intestine. The present invention provides the manner the orally intake calcium being absorbed through the intestine.

U.S. Pat. No. 5,880,109 discloses a method for accelerating intestinal absorption of calcium in a mammal in need of accelerated calcium absorption comprising orally administering a food, beverage, feed or pharmaceutical preparation to said mammal, wherein said food, beverage, feed or pharmaceutical preparation comprises water-soluble chitosan in an amount effective to accelerate intestinal absorption of calcium of said mammal.

U.S. Pat. No. 6,576,665 discloses the administering the calcium acetate composition, in the caplet-within-capsule form, to an individual to bind with the phosphorous in their gastrointestinal tract. Administration of the calcium acetate composition of the present invention according to the method described herein is associated with enhanced patient compliance and fewer side effects than is evident in administering presently available calcium acetate medications and phosphorus binders. This improved patient compliance with phosphate-binding ingestion will improve management of the disease process.

U.S. Pat. No. 6,582,722 discloses amino acid chelates for enhancing the absorption and assimilation of essential minerals in the human diet. Calcium, magnesium and potassium picolinic acid salts are disclosed as food and beverage supplements to improve the nutritive capacity of food stuffs and beverages

U.S. Pat. No. 6,984,377 discloses an oral care composition comprising a calcium metasilicate having an aspect ratio (average major axial diameter/average minor axial diameter) of from about 1:1 to about 2.5:, and an oil absorption of from about 20 ml/100 g to about 220 ml/100 g. Also disclosed is an oral care composition comprising: a calcium metasilicate having an aspect ratio (average major axial diameter/average minor axial diameter) of from about 1:1 to about 2.5:, and an oil absorption of from about 20 ml/100 g to about 220 ml/100 g, a disintegration aid, an organoleptic enhancing agent, and an abrasive.

U.S. Pat. No. 4,225,592 discloses method for treating a deficiency of at least one of the element selected from the group consisting of iron(II), copper(I), copper(II), magnesium(II), cobalt(II), manganese(II), zinc(II), chromium(III), molybdenum(V), vanadium(IV) and nickel(II), in humans, comprising administering to a human suffering from said deficiency an effective amount per day of said at least one of said elements plus potassium in the form of their complex formed with an oligo- or plygalacturonic acid wherein n is an integer from 10 to 145, M is said at least one metal cation selected from said group plus potassium, and Z is an integer corresponding to the charge or the valence number of the metal atom, said amount being effective to relieve said deficiency.

### SUMMARY OF THE INVENTION

It is an object of the present invention to provide an absorbable encapsulated calcium in hypromellose capsule, characterized in that the encapsulated calcium is being absorbed in vivo at the small intestine beyond the stomach, which maximize human absorption of calcium.

An object of the present invention is to provide an absorbable encapsulated calcium in hypromellose capsule, characterized in that the encapsulated calcium comprises calcium amino acid chelate extracted from soya bean, aspartate, calcium amino acid chelate obtained by chelation of aspartate with calcium hydroxide, collagen, lecithin, soya bean extracts, lactose, calcium carbonate, in the ratio of 4:8:8:10:8:12:10:40 being capsulated into hypromellose capsule capable of being absorbed in vivo at the small intestine beyond the stomach, which maximize human absorption of calcium.

Yet another object of the present invention is to provide a process for producing a calcium-containing capsule, wherein the process comprises the steps of preparing hypromellose capsule with delayed release of contents which are encapsulated; preparing calcium amino acid chelate and forming capsulated calcium capsule.

Another yet object of the present invention is to provide an absorbable encapsulated calcium in hypromellose capsule wherein the calcium salt is selected from the group consisting of calcium carbonate, and calcium acetate.

Still a further object of the present invention is to provide an absorbable encapsulated calcium in hypromellose capsule, wherein the chelated calcium amino acid is obtained from the extraction of soya bean.

Yet another object of the present invention is to provide an absorbable encapsulated calcium in hypromellose capsule, wherein the encapsulated calcium is from a calcium salt which is selected from the group consisting of calcium carbonate, calcium acetate, and wherein the encapsulated calcium is a water soluble chitosan.

### DETAILED DESCRIPTION OF AN ILLUSTRATIVE EMBODIMENT

The present invention takes advantage of the surprising discovery that calcium encapsulated in hypromellose capsules allows the active calcium to be released at a specific position with the intestine, enhancing the adsorption by the intestine.

In a first aspect of the invention, there is provided a controlled release formulation for use in oral dosage forms. The formulation is a mixture of hypromellose and calcium salt. The amount of mixture is an amount which is effective to provide controlled release of calcium being pharmaceutically active ingredient in vitro when the mixture is made into capsules.

In the present invention, the encapsulated calcium in hypromellose capsule, wherein the hypromellose provides a hydrophilic swellable structure capable of functioning as the gel layer while the calcium salt therein will be released when the hypromellose capsule is controlled to dissolve at the small intestine. In this way, the encapsulated calcium salt is controlled.

For purposes of the present invention, “targeted release” shall be understood to relate to the release of calcium salt to the small intestine from the mixure contained within a hypromellose capsule. As will be appreciated by those of ordinary skill, “targeted release” are not limited to extended or prolonged release profiles, it is to be understood that it is predictably after taking for a period of time.

The type of hypromellose included in the formulations of the present invention include all such types recognized in the art as being pharmaceutically acceptable. Hypromellose is also known in the art as hydroxypropylmethylcellulose (HPMC), and is a product from several chemical companies under different trade names. A non-limiting list of suitable HPMC includes for examples
(i) Enteric film; coating used on effervescent products (such as effervescent Vitamin C) to give immediate release of vitamins when in contact with liquid.
(ii) Extended time release; designed to slowly release vitamins over an extended period of time.
(iii) Controlled release; delivery of compounds in response to stimuli or time.

The advantages of hypromellose capsules are that they do not generally dissolve in gastric, which in turn , enter into the intestine to dissolve and release active ingredients.

The present invention relates to an orally administrable encapsulated calcium salt in hypromellose capsule.

The composition of the present invention preferably consists of calcium amino acid chelate (extracted from soya bean), aspartate, calcium amino acid chelate (chelated of aspartate with calcium amino acid), collagen, lecithin, extract of soya bean, lactose, calcium carbonate in the ratio of 4:8:8:10:8:12:10:40 being capsulated into hypromellose capsule. In the present invention, the amount of calcium amino chelate is high and it was obtained by two different methods such that the supplemental of calcium is comparatively balanced. The lecithin and the lactose added to the composition are for better absorption of calcium. Lactose increases adsorption rate of calcium within the small intestine. Lecithin is used to reduce fat so as to increase calcium adsorption. In food, the excessive fat contains fatty acid which will form into insoluble soapy calcium, and this will reduce calcium adsorption.

In the present preferred embodiment, the composition preferably consists of 60% calcium, and it is 0.5 g of calcium per capsule. The intake is 4 capsule per day, which is amounted to 1200 mg.

The present invention also relates to a method of enhancing intestinal absorption of calcium salt. More particularly, the method of the present invention facilitates the oral intake of calcium salt to effectively reduce stomach overcompensates for the high dose of calcium carbonate, which is alkaline, by churning out more acid. If the acid is not properly secreted, calcium salts are minimally dissolved (ionized) and, subsequently, it may not be properly and effectively absorbed. Additionally, the method of the present invention facilitates calcium absorption when the encapsulated calcium is consumed at four capsules per day.

The active ingredient of the soluble capsule is absorbed within the intestine, and the stimulus of pharmacologic drug is avoided, and the efficacy of the drug is increased and the side effect of the drug is decreased.

The calcium salt composition of the present invention is optionally dimensioned to form a tablet, a capsule or a caplet. Of these three forms, capsules are preferred as they completely being coated and enveloped the caplet until the capsule reaches the small intestine. The capsule at gastrointestinal tract, or stomach shall remain the same without being dissolved. Once the capsule is within the small intestine, the hypromellose capsule shell is dissolved and the medication is released for absorption.

Hypromellose includes the (i) intestinal hypromellose and (ii) colon hypromellose. The advantages of the intestinal hypromellose are that (i) avoid the stimulation of the drug within the stomach; (ii)avoid the unstable degradation of acid in the stomach, which improves the efficacies of the drug, (iii) control the location for the drug to be released, and (iv) avoid the degradation of the drug in the stomach. The hypromellose capsule is selected from the group consisting of Omeprazole Enteri-coated Capsules, and Aspirin Enteric Capsules. Any of the hypromellose capsule is applicable to the present invention.

The foregoing description has been directed to specific embodiments of this invention. It will be apparent, however, that other variations and modifications may be made to the described embodiments with the attainment of some or all of their advantages. Accordingly, this description should be taken only by way of example and not by way of limitation. It is the object of the appended claims to cover all such variations and modifications as come within the true spirit and scope of the invention.

What is claimed is:

1. An absorbable encapsulated calcium in hypromellose capsule, characterized in that the encapsulated calcium comprises calcium amino acid chelate extracted from soya bean, aspartate, calcium amino acid chelate which is obtained from chelation of aspartate with calcium hydroxide, collagen, lecithin, soya bean extract, lactose, calcium carbonate, in the ratio of 4:8:8:10:8:12:10:40 being capsulated into hypromellose capsule capable of being absorbed in vivo at the small intestine beyond the stomach, which maximize human absorption of calcium.

2. The absorbable encapsulated calcium in hypromellose capsule of claim 1, wherein the encapsulated calcium is from a calcium salt.

3. The absorbable encapsulated calcium in hypromellose capsule of claim 2, wherein the calcium salt is selected from the group consisting of calcium carbonate, and calcium acetate.

4. The absorbable encapsulated calcium in hypromellose capsule of claim of claim 3, wherein the encapsulated calcium is a water soluble chitosan.

5. A calcium-containing capsule as defined in claim 2, wherein the calcium salt is a salt of an organic carboxylic acid.

6. A process for producing a calcium-containing capsule, wherein the process comprises the steps of preparing hypromellose capsule with delayed release of contents which are encapsulated; preparing calcium amino acid chelate and forming capsulated calcium capsule.

7. The process as set forth in claim 6, wherein the chelated calcium amino acid is obtained from the extraction of soya bean.

8. The process as set forth in claim 6, wherein the chelated calcium amino acid is obtained by the method of chelation of aspartate and calcium hydroxide and added with aspartate calcium and calcium carbonate.

9. The absorbable encapsulated calcium in hypromellose capsule as set forth in claim 1, wherein the calcium content is about 60%.

10. The calcium-containing capsule as set forth in claim 7, wherein the calcium content is about 60%.

11. The absorbable encapsulated calcium in hypromellose capsule as set forth in claim 1, wherein the hypromellose capsule is selected from the group consisting of Omeprazole Enteri-coated Capsules, and Aspirin Enteric Capsules.

12. The calcium-containing capsule as set forth in claim 5, wherein the hypromellose capsule is selected from the group consisting of Omeprazole Enteri-coated Capsules and Aspirin Enteric Capsules.
